(19) United States
(12) Patent Application Publication
(10) Pub. No.: US 2017/0165006 A1
(43) Pub. Date: Jun. 15, 2017

(54) Tracking Devices For Use In Navigation Systems And Methods For Manufacturing The Same

(71) Applicant: Stryker Corporation, Kalamazoo, MI (US)

(72) Inventors: Philip Robert Woods, Kalamazoo, MI (US); Christopher Donald Philipp, Portage, MI (US)

(73) Assignee: Stryker Corporation, Kalamazoo, MI (US)

(21) Appl. No.: 15/370,262

(22) Filed: Dec. 6, 2016

Related U.S. Application Data
(60) Provisional application No. 62/265,585, filed on Dec. 10, 2015.

(51) Int. Cl. A61B 34/20

(52) U.S. Cl. CPC A61B 34/20; A61B 2034/2055

(57) ABSTRACT

Tracking devices for use in a navigation system and methods of manufacturing the same. The tracking device comprises a tracking head and a plurality of emitters supported by the tracking head to emit light. A plurality of dome-shaped lenses having arcuate inner and outer surfaces define an outer wall of uniform thickness disposed over the emitters so that light is emitted through the outer wall in a manner that minimizes light refraction.

### RELATED APPLICATIONS

This application claims the benefit of and priority to U.S. Provisional Patent Application No. 62/265,585, filed Dec. 10, 2015, the entire contents and disclosure of which are hereby incorporated by reference herein.

### TECHNICAL FIELD

The disclosure relates generally to tracking devices used in navigation systems and methods for manufacturing the same.

### BACKGROUND

Navigation systems assist users in locating objects. For instance, navigation systems are used in industrial, aerospace, and medical applications. In the medical field, navigation systems assist surgeons in locating surgical instruments and anatomy for the purpose of accurately placing the surgical instruments relative to the anatomy. Typically, the surgical instruments and the anatomy are tracked together with their relative movement shown on a display.

Navigation systems may employ light signals, sound waves, magnetic fields, radio frequency signals, etc. in order to track the position and/or orientation of objects. Often the navigation system includes tracking devices attached to the objects being tracked. A localizer cooperates with tracking elements on the tracking devices to determine positions of the tracking elements, and ultimately to determine a position and orientation of the objects. The navigation system monitors movement of the objects via the tracking devices.

Some navigation systems employ active tracking elements that directly emit light to be received by the localizer to triangulate the positions of the tracking elements. One advantage of employing active tracking elements is increased accuracy as compared to other navigation systems, such as navigation systems that rely on reflector elements that reflect light emitted by the localizer. In navigation systems that employ active tracking elements, the tracking elements are usually located behind transparent lenses, which enable the tracking devices to be sterilized for reuse, but which still allow the light to be emitted from the tracking elements to the localizer. However, these transparent lenses can cause refraction in the light being emitted from the tracking elements. This refraction can result in the localizer inaccurately determining positions of the tracking elements thereby resulting in an inaccurate calculation of the position and orientation of the object being tracked. Aside from reducing refraction, in order to realize suitable accuracy in some applications, the tracking elements must be placed with high precision during manufacturing, which is difficult to accomplish in some cases. When imprecise manufacturing is combined with issues associated with refraction of the light, accuracy errors can become undesirable.

As a result, there is a need in the art for tracking devices that overcome one or more of the problems mentioned above.

### SUMMARY

In one embodiment, a tracking device is provided for use with a navigation system to track an object. The tracking device comprises a tracking head. A plurality of emitters are supported by the tracking head. Each of the emitters is configured to emit light. A plurality of lenses are disposed over the plurality of emitters. Each of the lenses has an outer wall with an arcuate inner surface and an arcuate outer surface spaced equidistantly from the arcuate inner surface to define a uniform outer wall thickness. The lenses are arranged relative to the emitters such that the light emitted from the emitters penetrate the outer wall normal to the arcuate inner surface to minimize refraction of the light.

In another embodiment, a navigation system is provided for tracking an object. The navigation system comprises a tracking head. A plurality of emitters are supported by the tracking head. Each of the emitters is configured to emit light. A plurality of lenses are disposed over the plurality of emitters. Each of the lenses has an outer wall with an arcuate inner surface and an arcuate outer surface spaced equidistantly from the arcuate inner surface to define a uniform outer wall thickness. The lenses are arranged relative to the emitters such that the light emitted from the emitters penetrate the outer wall normal to the arcuate inner surface to minimize refraction of the light. The navigation system also comprises a localizer for receiving the light emitted from the emitters to determine a position and orientation of the object.

A method of manufacturing a tracking device is also provided. The method comprises assembling a plurality of emitters to a base at a predefined z-axis height relative to the base. Each of the emitters is configured to emit light. The method further comprises locating a plurality of lenses over the plurality of emitters. Each of the lenses has an outer wall with an arcuate inner surface and an arcuate outer surface spaced equidistantly from the arcuate inner surface to define a uniform outer wall thickness. Each of the plurality of lenses are located relative to the emitters in an x-y plane such that the light emitted from the emitters penetrate the outer wall normal to the arcuate inner surface to minimize refraction of the light.

### BRIEF DESCRIPTION OF THE DRAWINGS

Advantages of the present disclosure will be readily appreciated as the same becomes better understood by reference to the following detailed description when considered in connection with the accompanying drawings wherein:

FIG. 1 is a perspective view of a navigation system being used in conjunction with a patient;

FIG. 2 is a schematic view of the navigation system;

FIG. 3 is a perspective view of a tracking device;

FIG. 4 is a cross-sectional view of the tracking device;

FIG. 5 is a perspective view of a first housing of the tracking device;

FIG. 6 is an exploded view of the first housing of the tracking device;

FIG. 7 is a top perspective of an emitter support assembly;

FIG. 8 is a bottom perspective of the emitter support assembly;

FIG. 9 is a top perspective view of the first housing with the emitter support assemblies;

FIG. 10 is a bottom perspective view of the first housing with the emitter support assemblies;

FIG. 11 is a top perspective view of the first housing with the emitter support assemblies, light emitting diodes, and lenses;

FIG. 12 is a perspective view of one of the lenses;

FIG. 13 is a cross-sectional view of one of the emitter support assemblies and one of the lenses;

FIG. 14 is a graph illustrating errors in a LED that is fired as it is rotated around a circle on a rotary turntable;

FIG. 15 is a bottom perspective view of a second housing of the tracking device;

FIG. 16 is a top perspective view of the second housing of the tracking device;

FIG. 17 is a bottom perspective view of a connector of the tracking device;

FIG. 18 is an exploded view of the connector of the tracking device;

FIG. 19 is a top perspective view of the connector;

FIG. 20 is a perspective and cross-sectional view of the connector attached to the second housing;

FIG. 21 is a bottom perspective view of the connector attached to the second housing with a mating cable connector;

FIG. 22 is a top perspective view of the connector attached to the second housing;

FIG. 23 is a top perspective view of magnets attached to the connector; and

FIG. 24 is a top perspective view of a flux return plate located adjacent to the magnets.

### DETAILED DESCRIPTION

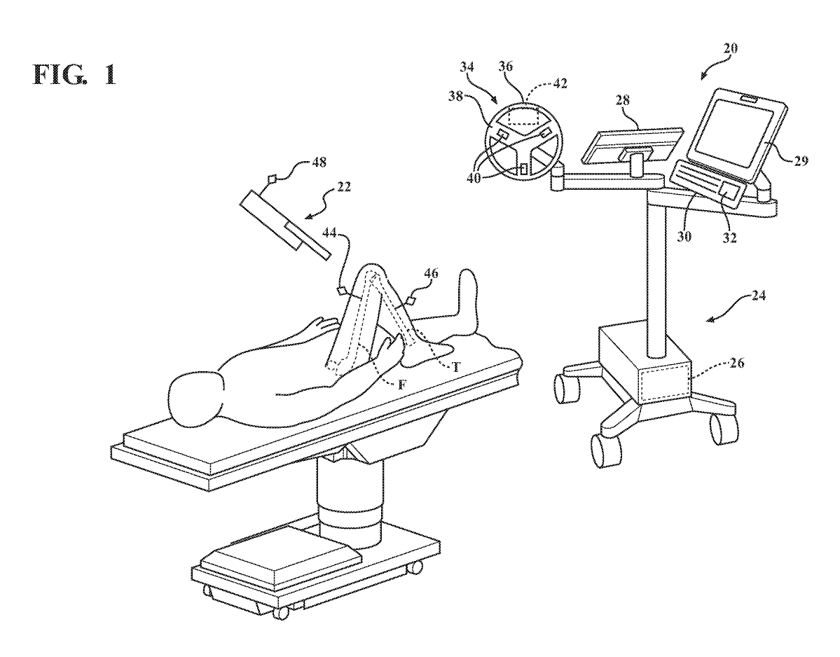

Referring to FIG. 1 a navigation system 20 is illustrated. The navigation system 20 is shown in a surgical setting such as an operating room of a medical facility. The navigation system 20 is set up to track movement of various objects in the operating room. Such objects may include, for example, a surgical instrument 22, a femur F of a patient, and a tibia T of the patient. The navigation system 20 tracks these objects for purposes of displaying their relative positions and orientations to the surgeon and, in some cases, for purposes of controlling or constraining movement of the surgical instrument 22 relative to a predefined path or anatomical boundary.

The navigation system 20 includes a computer cart assembly 24 that houses a navigation computer 26. A navigation interface is in operative communication with the navigation computer 26. The navigation interface includes a first display 28 adapted to be situated outside of a sterile field and a second display 29 adapted to be situated inside the sterile field. The displays 28, 29 are adjustably mounted to the computer cart assembly 24. First and second input devices 30, 32 such as a mouse and keyboard can be used to input information into the navigation computer 26 or otherwise select/control certain aspects of the navigation computer 26. Other input devices are contemplated including a touch screen (not shown) on the displays 28, 29 or voice-activation.

A localizer 34 communicates with the navigation computer 26. In the embodiment shown, the localizer 34 is an optical localizer and includes a camera unit 36 (also referred to as a sensing device). The camera unit 36 has an outer casing 38 that houses one or more optical position sensors 40. In some embodiments at least two optical sensors 40 are employed, sometimes three or four. The optical sensors 40 may be three separate charge-coupled devices (CCD). In one embodiment three, one-dimensional CCDs are employed. It should be appreciated that in other embodiments, separate camera units, each with a separate CCD, or two or more CCDs, could also be arranged around the operating room. The CCDs detect infrared (IR) light signals.

Camera unit 36 is mounted on an adjustable arm to position the optical sensors 40 with a field of view of the below discussed trackers that, ideally, is free from obstructions.

The camera unit 36 includes a camera controller 42 in communication with the optical sensors 40 to receive signals from the optical sensors 40. The camera controller 42 communicates with the navigation computer 26 through either a wired or wireless connection (not shown). One such connection may be an IEEE 1394 interface, which is a serial bus interface standard for high-speed communications and isochronous real-time data transfer. The connection could also use a company specific protocol. In other embodiments, the optical sensors 40 communicate directly with the navigation computer 26.

Position and orientation signals and/or data are transmitted to the navigation computer 26 for purposes of tracking the objects. The computer cart assembly 24, the display 28, and the camera unit 36 may be like those described in U.S. Pat. No. 7,725,162 to Malackowski, et al. issued on May 25, 2010, entitled “Surgery System,” hereby incorporated by reference.

The navigation computer 26 can be a personal computer or laptop computer. Navigation computer 26 has the displays 28, 29, central processing unit (CPU) and/or other processors, memory (not shown), and storage (not shown). The navigation computer 26 is loaded with software as described below. The software converts the signals received from the camera unit 36 into data representative of the position and orientation of the objects being tracked.

Navigation system 20 includes a plurality of tracking devices 44, 46, 48, also referred to herein as trackers. In the illustrated embodiment, one tracker 44 is firmly affixed to the femur F of the patient and another tracker 46 is firmly affixed to the tibia T of the patient. Trackers 44, 46 are firmly affixed to sections of bone. Trackers 44, 46 may be attached to the femur F and tibia T in the manner shown in U.S. Pat. No. 7,725,162, hereby incorporated by reference and/or in the manner shown in U.S. patent application Ser. No. 14/156,856, filed Jan. 16, 2014, entitled, “Navigation Systems and Methods for Indicating and Reducing Line-of-Sight Errors” and published as U.S. Patent Application Publication No. 2014/0200621, hereby incorporated by reference herein. Other methods of attachment are also possible. In additional embodiments, a tracker (not shown) is attached to the patella to track a position and orientation of the patella. In yet further embodiments, the trackers 44, 46 could be mounted to other tissue types or parts of the anatomy.

An instrument tracker 48 is firmly attached to the surgical instrument 22. The instrument tracker 48 may be integrated into the surgical instrument 22 during manufacture or may be separately mounted to the surgical instrument 22 in preparation for the surgical procedure. The working end of the surgical instrument 22, which is being tracked, may be a rotating bur, electrical ablation device, other energy applicators, or the like.

In the embodiment shown, the surgical instrument 22 may be an end effector attached to a surgical manipulator. Such an arrangement is shown in U.S. Pat. No. 9,119,655, granted Sep. 1, 2015, entitled, “Surgical Manipulator Capable of Controlling a Surgical Instrument in Multiple Modes,” the disclosure of which is hereby incorporated by reference.

In other embodiments, the surgical instrument 22 may be manually positioned by only the hand of the user, without the aid of any cutting guide, jib, or other constraining mechanism such as a manipulator or robot. Such a surgical instrument is described in U.S. patent application Ser. No. 13/600,888, filed Aug. 31, 2012, entitled, “Surgical Instrument Including Housing, a Cutting Accessory that Extends from the Housing and Actuators that Establish the Position of the Cutting Accessory Relative to the Housing”, the disclosure of which is hereby incorporated by reference.

The optical sensors 40 of the localizer 34 receive light signals from the trackers 44, 46, 48. In the illustrated embodiment, the trackers 44, 46, 48 are active trackers. In this embodiment, each tracker 44, 46, 48 has at least three, and preferably four, active tracking elements for transmitting light signals to the optical sensors 40. The tracking elements can be, for example, light emitting diodes (LEDs) 50 transmitting light, such as infrared light. The optical sensors 40 preferably have sampling rates of 100 Hz or more, more preferably 300 Hz or more, and most preferably 500 Hz or more. In some embodiments, the optical sensors 40 have sampling rates of 8000 Hz. The sampling rate is the rate at which the optical sensors 40 receive light signals from sequentially fired LEDs 50. In some embodiments, the light signals from the LEDs 50 are fired at different rates for each tracker 44, 46, 48.

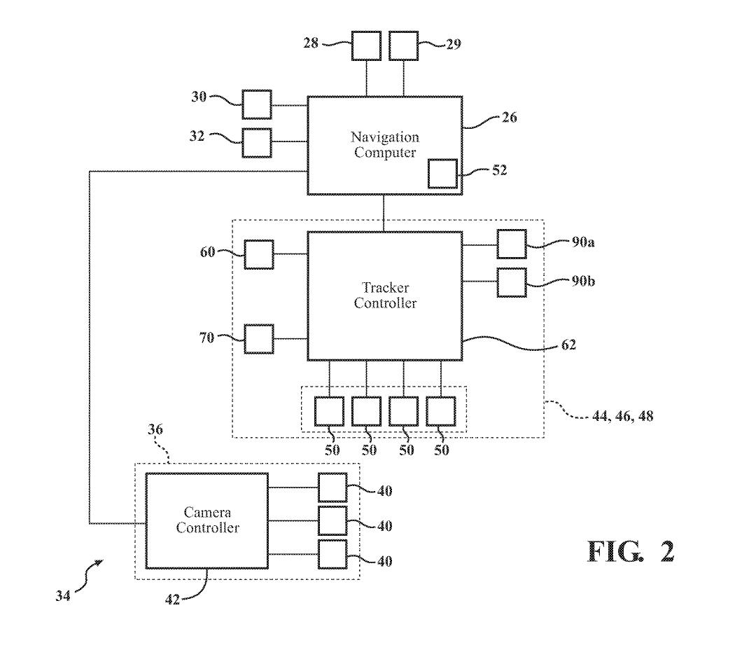

Referring to FIG. 2, each of the LEDs 50 are connected to a tracker controller 62 of the associated tracker 44, 46, 48 that transmits/receives data to/from the navigation computer 26. In one embodiment, the tracker controllers 62 transmit data on the order of several Megabytes/second through wired connections with the navigation computer 26. In other embodiments, a wireless connection may be used. In these embodiments, the navigation computer 26 has a transceiver (not shown) to receive the data from the tracker controller 62.

Each of the trackers 44, 46, 48 also includes a 3-dimensional gyroscope sensor 60 that measures angular velocities of the trackers 44, 46, 48. The angular velocities measured by the gyroscope sensors 60 provide additional non-optically based data for the navigation system 20 with which to track the trackers 44, 46, 48. Each of the trackers 44, 46, 48 also includes a 3-axis accelerometer 70. The accelerometers 70 provide additional non-optically based data for the navigation system 20 with which to track the trackers 44, 46, 48.

Each of the gyroscope sensors 60 and accelerometers 70 communicate with the tracker controller 62 located in the housing of the associated tracker that transmits/receives data to/from the navigation computer 26. The data can be received either through a wired or wireless connection.

The navigation computer 26 includes a navigation processor 52. The camera unit 36 receives optical signals from the LEDs 50 of the trackers 44, 46, 48 and outputs to the processor 52 signals and/or data relating to the position of the LEDs 50 of the trackers 44, 46, 48 relative to the localizer 34, which can be signals and/or data determined by triangulation. The gyroscope sensors 60 transmit non-optical signals to the processor 52 relating to the 3-dimensional angular velocities measured by the gyroscope sensors 60. Based on the received optical and non-optical signals, navigation processor 52 generates data indicating the relative positions and orientations of the trackers 44, 46, 48 relative to the localizer 34. Trackers without gyroscope sensors or accelerometers could also be employed.

It should be understood that the navigation processor 52 could include one or more processors to control operation of the navigation computer 26. The processors can be any type of microprocessor or multi-processor system, or other types of processors. The term processor is not intended to be limited to a single processor.

Prior to the start of the surgical procedure, additional data are loaded into the navigation processor 52. Based on the position and orientation of the trackers 44, 46, 48 and the previously loaded data, navigation processor 52 determines the position of the working end of the surgical instrument 22 and the orientation of the surgical instrument 22 relative to the tissue against which the working end is to be applied.

The navigation processor 52 also generates image signals that indicate the relative position of the surgical instrument working end to the surgical site. These image signals are applied to the displays 28, 29. Displays 28, 29, based on these signals, generate images that allow the surgeon and staff to view the relative position of the surgical instrument working end to the surgical site. The displays, 28, 29, as discussed above, may include a touch screen or other input/output device that allows entry of commands.

In some embodiments, only one LED 50 can be read by the optical sensors 40 at a time. The camera controller 42, through one or more infrared or RF transceivers (on camera unit 36 and trackers 44, 46, 48), or through a wired connection, may control the firing of the LEDs 50, as described in U.S. Pat. No. 7,725,162 to Malackowski, et al., hereby incorporated by reference. Alternatively, the trackers 44, 46, 48 may be activated locally (such as by a switch on trackers 44, 46, 48) which then fires its LEDs 50 sequentially once activated, without instruction from the camera controller 42.

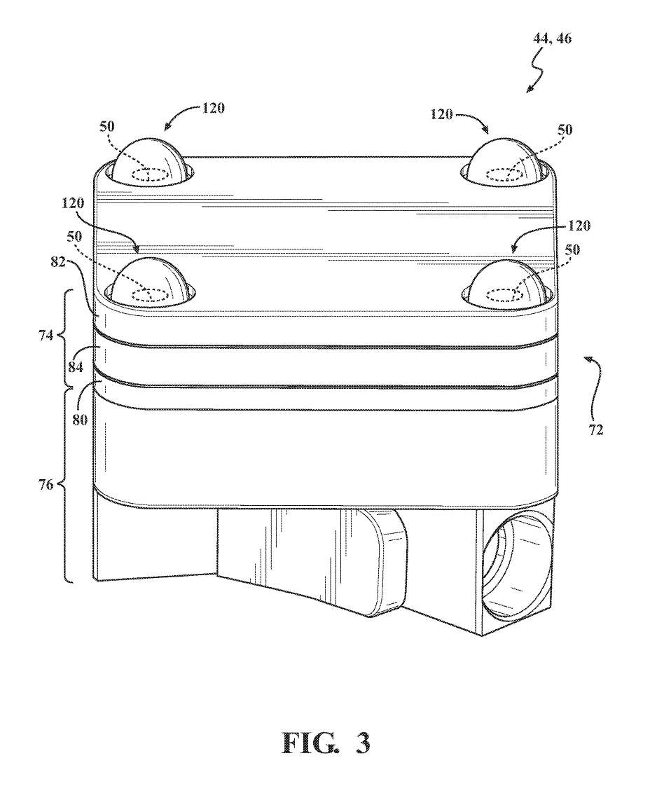

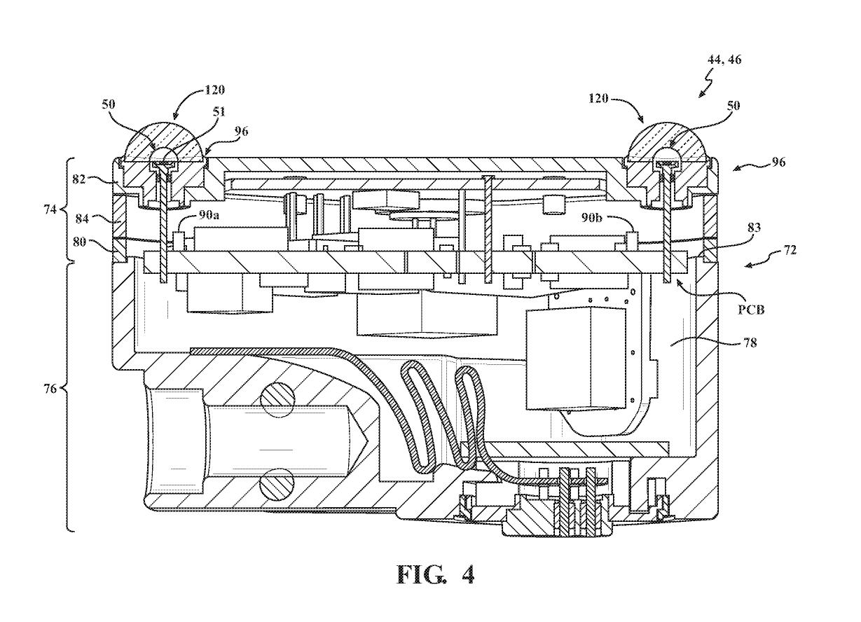

One version of the trackers 44, 46 attached to the femur F and tibia T is shown in FIGS. 3 and 4. For simplicity, reference will be made below to only one of the trackers 44, 46, but the trackers 44 and 46 may be identical.

The tracker 44 comprises a tracking head 72 configured to be mounted to an object for purposes of tracking a position and orientation of the object in accordance with the principles described above. Four tracking elements are provided for use by the navigation system 20 to track the position and orientation of the object. In the version shown, the four tracking elements are in the form of infrared LEDs 50. The LEDs 50 are supported by the tracking head 72 to emit light that will be received by the optical sensors 40 of the localizer 34.

The tracking head 72 comprises a first housing 74 and a second housing 76. The second housing 76 is connected to the first housing 74 to define an internal chamber 78. The first housing 74 and the second housing 76 have congruent outer surfaces to form a continuous outer surface of the tracking head 72. The continuous outer surface may have a generally rectangular shape, with rounded corners, as best shown in FIG. 3.

A printed circuit board (PCB), upon which is mounted the tracker controller 62, the gyroscope 60, the accelerometer 70, and other internal electronic components, is disposed in the internal chamber 78 and hermetically sealed inside the internal chamber 78 to allow for sterilization of the tracker 44 without concern for damaging these electronic components disposed in the internal chamber 78.

As shown in FIGS. 3-6, the first housing 74 comprises a weld ring 80, a lid 82, and a light ring 84. The weld ring 80, lid 82, and light ring 84, have congruent outer surfaces to form part of the continuous outer surface of the tracking head 72.

The weld ring 80 is formed of titanium. The weld ring 80 is configured to be laser welded to the second housing 76, which may also be formed of titanium. The weld ring 80 has a generally uniform thickness about its periphery and is ring shaped. During manufacture, the weld ring 80 is seated on a shoulder of the second housing 76 and welded thereto. In the embodiment shown, the weld ring 80 has a plurality of positioning projections 81. The projections 81 are located centrally on an inner surface of each side of the weld ring 80. The projections 81 span only a portion of the inner surface and project inwardly from the inner surface to ultimately rest on a peripheral rim 83 of the second housing 76 during assembly (see FIGS. 4 and 6). In other embodiments, the projections 81 may be replaced by a continuous, peripheral flange that rests on the rim 83 of the second housing 76.

The lid 82 is formed of titanium. The LEDs 50 are arranged to emit light beyond the lid 82. The light ring 84 is disposed between the weld ring 80 and the lid 82. The light ring 84 may be formed of zirconia or other materials that illuminate to indicate certain status conditions of the tracker 44 and/or other components/systems during use. The weld ring 80 and the lid 82 are brazed to the light ring 84 using brazing preforms 86 during manufacture. The weld ring 80 and the light ring 84 may have the same outer shape and thickness.

At least one status indicating light source is arranged to emit light through the light ring 84. The at least one indicating light source may comprise: an LED; a plurality of multi-color LEDs, such as RGB LEDs, each capable of emitting light of different colors; a first plurality of LEDs capable of emitting light of a first color and a second plurality of LEDs capable of emitting light of a second color, different than the first color; or any combination of these. In one case, the status indicating light source comprises at least one LED 90a capable of emitting orange visible light and at least one LED 90b capable of emitting green visible light.

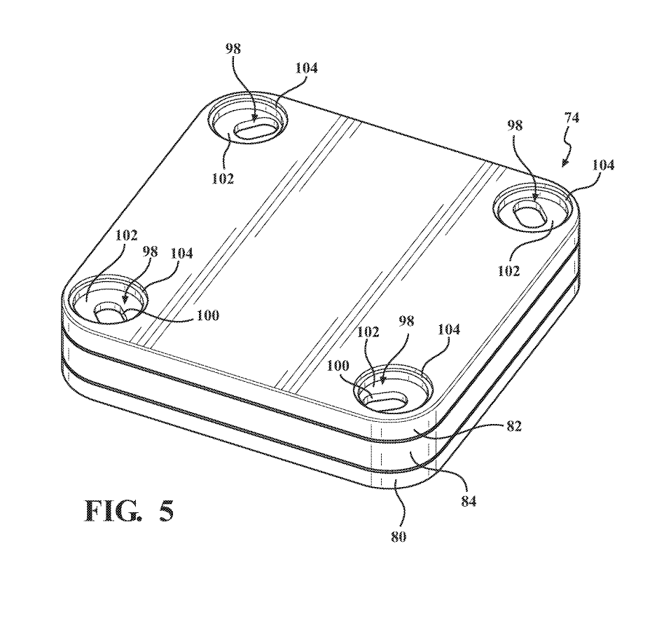

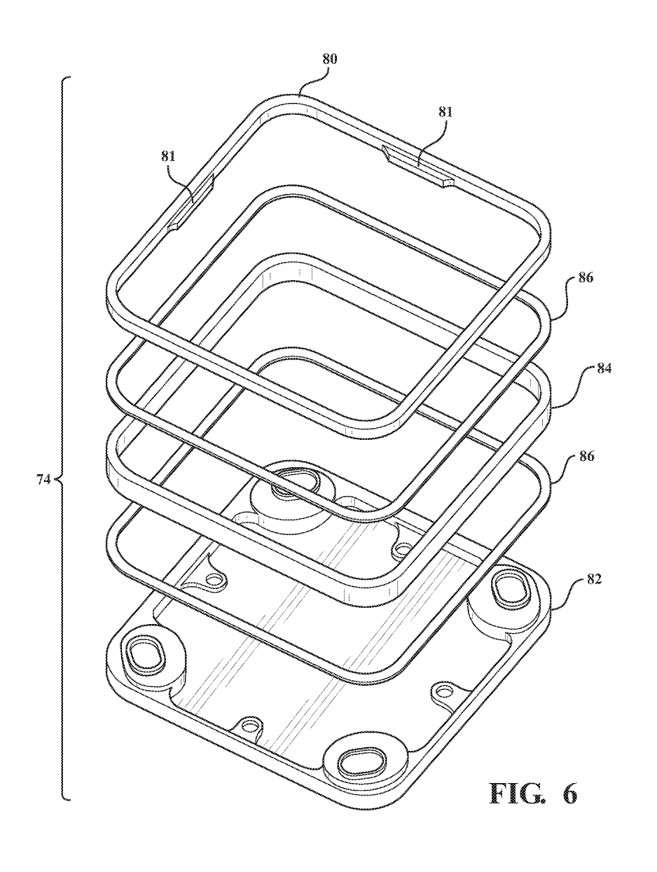

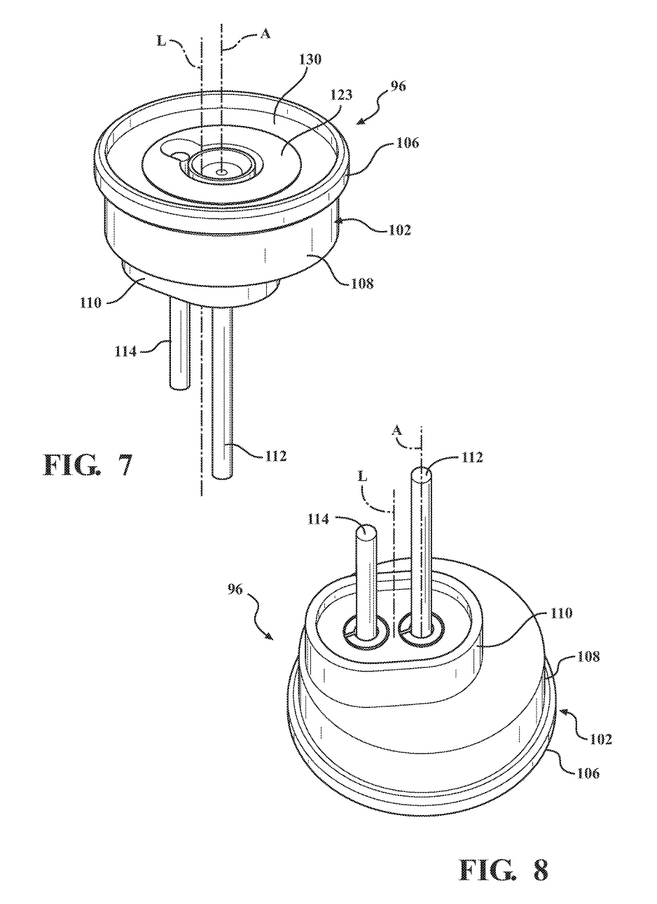

Referring to FIGS. 4-8, a plurality of emitter support assemblies 96 are supported in the lid 82 of the first housing 74 to support the LEDs 50. In particular, the lid 82 defines a plurality of pockets 98 (see FIG. 5) for receiving the emitter support assemblies 96. Each of the pockets 98 has a through opening 100, an inner counterbore 102, and an outer counterbore 104. It should be appreciated that the through opening 100 and the counterbores 102, 104 may be formed by machining, molding/casting or otherwise.

Each of the emitter support assemblies 96 comprises a base 102. The base 102 may be formed of metal, such as 304L stainless steel. The base 102 comprises an annular rim 106 that is fixed to the lid 82 and rests in the outer counterbore 104. The base 102 also comprises a cylindrical body 108 depending downwardly from the rim 106. The body 108 is fixed to the lid 82 and rests in the inner counterbore 102. The base 102 also comprises an alignment section 110 that depends downwardly from the body 108.

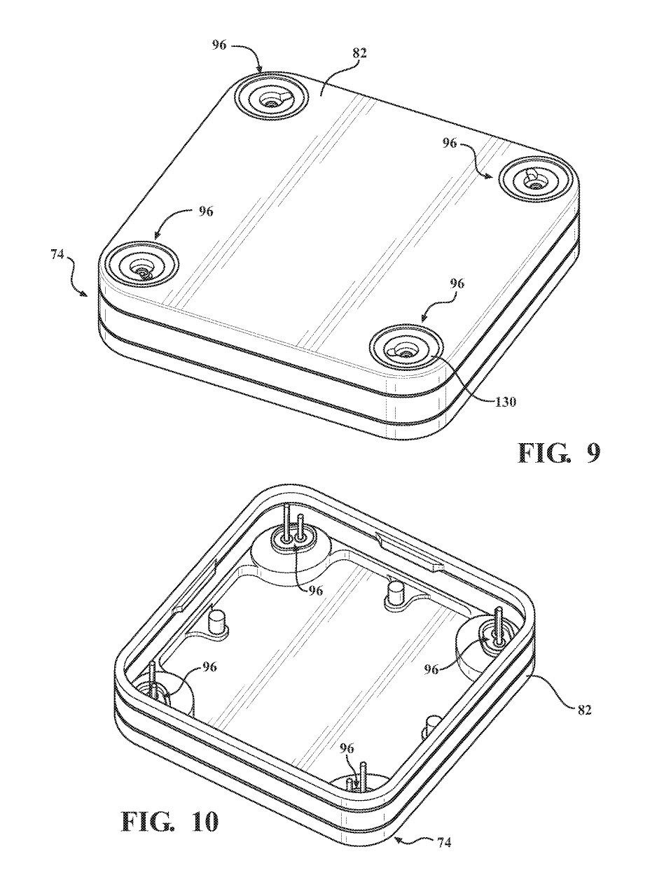

The alignment section 110 has a longitudinal axis L that is offset from a central axis A of the body 108. The alignment section 110 has an oblong shape so that the emitter support assembly 96 is only able to be fitted in the pocket 98 in one orientation during manufacture. The emitter support assemblies 96 are fixed to the lid 82 by one or more of brazing, laser welding, adhesive, or the like. Attachment of the emitter support assemblies 96 is shown in FIGS. 9 and 10.

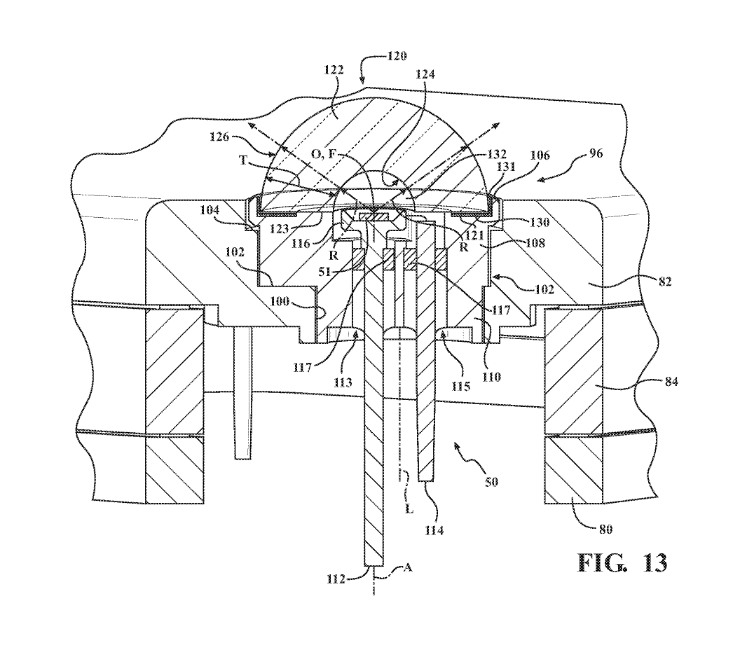

Each of the emitter support assemblies 96 also comprise a pair of pins 112, 114 located in through openings 113, 115 (see FIG. 13) in the base 102. The pins 112, 114 may be formed at least partially of copper beryllium and, in some cases, completely of copper beryllium, other copper alloys, or other materials. The pins 112, 114 may be fixed and sealed to the base 102 inside the through openings 113, 115 with polycrystalline ceramic 117, such as Kryoflex (see FIG. 13). The pins 112, 114 provide the cathode and anode contacts for the LEDs 50.

A reflector head 116 is integral with the pin 112, such that the reflector head 116 and the pin 112 are formed as one piece. An emitter of each LED 50 is supported by and centered relative to each reflector head 116. The emitter, in one embodiment, is a die 51 of the LED 50. In one exemplary version, the die 51 is an EPIGAP™ die from Optoelektronik GmbH, D-12555 Berlin, Kopenicker Str. 325 b, Haus 201, serial no. ELC-875-22.

The reflector head 116 is configured to contact the die 51 so that the reflector head 116 provides connection to the anode contact of the LED 50 through an anode connection. The cathode connection is provided by contact with the die 51 at a center of the pin 112. The reflector head 116 may be cup-shaped or any shape suitable for supporting the die 51. The die 51 may be secured to the reflector head 116 by an adhesive and coated with silicon gel or other coating. In some embodiments, the die 51 may be covered with silicon gel to encapsulate the die 51 in the reflector head 116 (not shown).

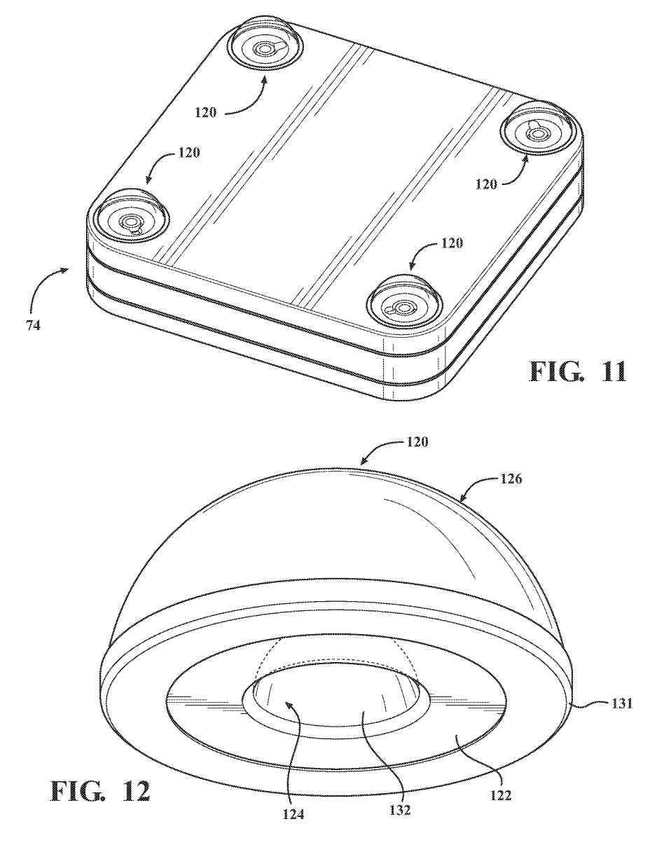

Referring to FIGS. 11-14, a lens 120 is disposed over each of the dies 51 seated in the reflector head 116 such that each of the dies 51 emits light through a corresponding one of the lenses 120 to the optical sensors 40. Each of the lenses 120 has an arcuate outer wall 122 with an arcuate inner surface 124 and an arcuate outer surface 126. The arcuate outer surface 126 is spaced equidistantly from the arcuate inner surface 124 to define a uniform wall thickness T. Each of the lenses 120 are arranged relative to the dies 51 such that light rays R emitted from a focal point F of the dies 51 penetrate the outer wall 122 normal to the arcuate inner surface 124 to minimize refraction of the light rays. Each of the lenses 120 has a generally domed shape and are formed of sapphire.

The lenses 120 are soldered to the emitter support assemblies 96. More specifically, each of the lenses 120 is soldered to the base 102 with a solder ring 130. The solder ring 130 has a flat ring portion and an upwardly extending lip portion. The flat ring portion is sized to fit within an upper, annular groove 121 formed in an upper surface of the base 102. The groove 121 is located between an inner raised boss 123 of the base and the rim 106. The solder ring 130, when heated, secures the lens 120 to the base 102 about the reflector head 116 and within the rim 106.

Owing to the arcuate shape of the outer wall 122 of the lens 120, each of the lenses 120 defines an inner space 132 for receiving the die 51. As a result, the dies 51 can be arranged so that the light rays R emitted from the focal points F of the dies 51 impact the outer wall 122 normal to the outer wall 122 to minimize refraction of the light rays. More specifically, the outer wall 122 defines a hemisphere having a geometric centroid O. The focal point F of the die 51 is coincident with the centroid O of the hemisphere so that the light rays R emitted from the focal point F of the die 51 travel the same distance in all directions to reach the outer wall 122. This geometric arrangement results in the die 51 being realized as a point light source to the localizer 34.

An antireflective coating is applied to each of the lenses 120 during manufacture. The coating may be sputter coated onto the lenses 120 or applied by other conventional methods. The antireflective coating is designed for the infrared spectrum and may be applied on the inner and/or outer surfaces 124, 126.

During manufacture, each of the dies 51 is assembled so that the focal point F of the die 51 is coincident with the centroid O of the lens 120. In one embodiment, the reflector head 116, preferably before the die 51 is secured therein (but in some cases after), is inserted into the base 102 such that the reflector head 116 is positioned at a predefined z-axis height relative to the base 102. The reflector head 116 is then welded or brazed and sealed to the base 102. Accordingly, owing to an insignificant tolerance in the die 51, when the die 51 is thereafter positioned and fixed in the reflector head 116, the die 51 will be appropriately positioned with respect to the z-axis. In other embodiments, the die 51 may be first positioned and fixed in the reflector head 116 and then optically tracked and measured by an optical measuring device (not shown), until the focal point F of the die 51 is at a predefined z-axis height with the reflector head (and carried die 51) being fixed to the base 102.

Once the die 51 is fixed in position in the reflector head 116, the lens 120 is placed over the die 51. The z-axis height of the lens 120 is set by virtue of a bottom of the lens 120 abutting the boss 123 on the base 102. The lens 120 also needs to be positioned with respect to the x-y plane so that the light rays R emitted from the focal point F of the die 51 penetrate the outer wall 122 of the lens 120 normal to the arcuate inner surface 124 to minimize refraction of the light rays R. During placement of the lens 120, the lens 120 is optically tracked and measured by an optical measuring device to ensure that the lens 120 is centered in the x-y plane relative to the die 51. By centering the lens 120 in the x-y plane relative to the die 51, the centroid of the lens 120 becomes coincident with the focal point F of the die 51.

Once the lens 120 is in position, the lens 120 is soldered to the base 102 using the solder ring 130. In the version shown in FIG. 13, the bottom of the lens 120 rests on the boss 123 above the groove 121. This placement rigidly controls the z-axis height of the lens 120 on the base 102, as previously described. With the lens 120 then held in place on the boss 123, the solder ring 130 is heated and liquefied to fix an outer edge of the lens 120 to the rim 106. Solder material also flows beneath the lens 120 to fix the bottom of the lens 120 to the base 102. In some embodiments, a metalized coating 131 may be applied to the bottom and partially along the outer surface 126 of the lens 120 to facilitate better soldering of the lens 120 to the base 102.

Special fixtures can be created to repeatably place the reflector heads 116 at the predefined z-axis height relative to the bases 102 and to repeatably place the lenses 120 so that the focal points F of the dies 51 are located at the geometric centroid O of the hemisphere. In particular, a fixture may receive the base 102, the pins 112, 114 (including the reflector head 116), the solder ring 130, the lens 120, and the polycrystalline ceramic 117 between the pins 112, 114 and the base 102. The fixture may be designed to establish and hold desired heights between these parts when placed in the fixture. The optical measuring device determines the appropriate x-y positioning of the lens 120. Once positioned, the lens 120 can also be held in place in the fixture with respect to the die 51. These parts are then heated so that the solder ring 130 melts to fix the metalized coating 131 of the lens 120 to the base 102 and the polycrystalline ceramic melts to fix the pins 112, 114 (and the reflector head 116) to the base 102 at the predefined height.

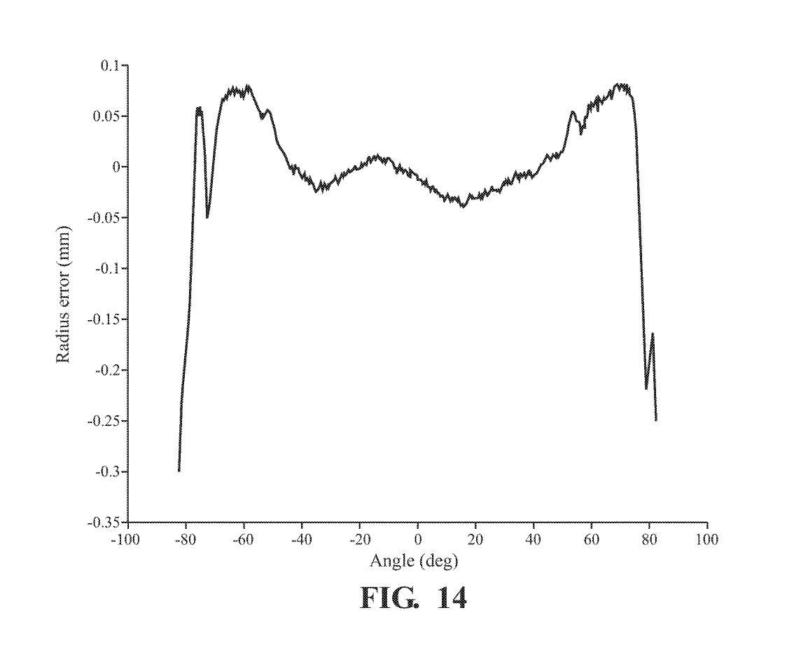

FIG. 14 illustrates a plot of an exemplary LED that was tested on a rotary turntable. Data was collected by the camera unit 36 as the LED was fired while the LED rotated around a circle. A circle fit was applied to this data to determine position error over a viewing angle of the LED. As shown, there is basically no error at zero degrees and, as the LED sweeps through the viewing angle, at 80 degrees there is a worst case error of approximately 0.1 mm. In other cases, the error is unable to be measured by the camera unit 36 as the error is within a noise floor of the camera unit 36, e.g., the error is less than +/−0.05 mm of position error.

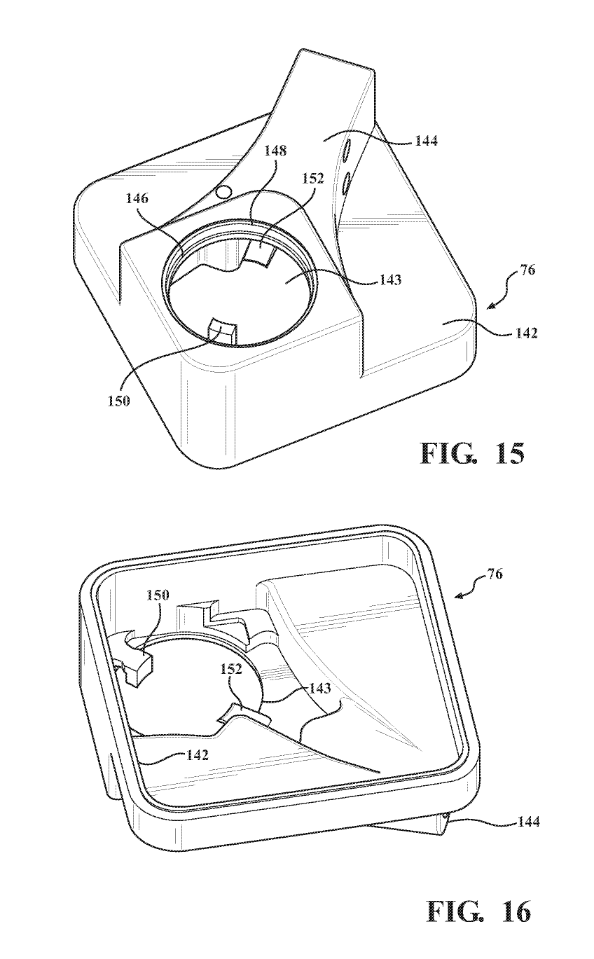

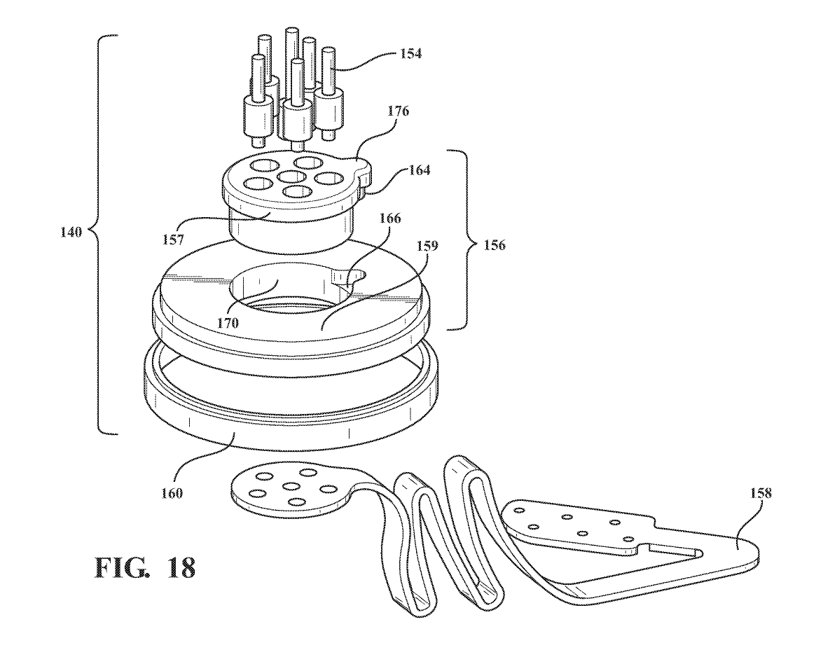

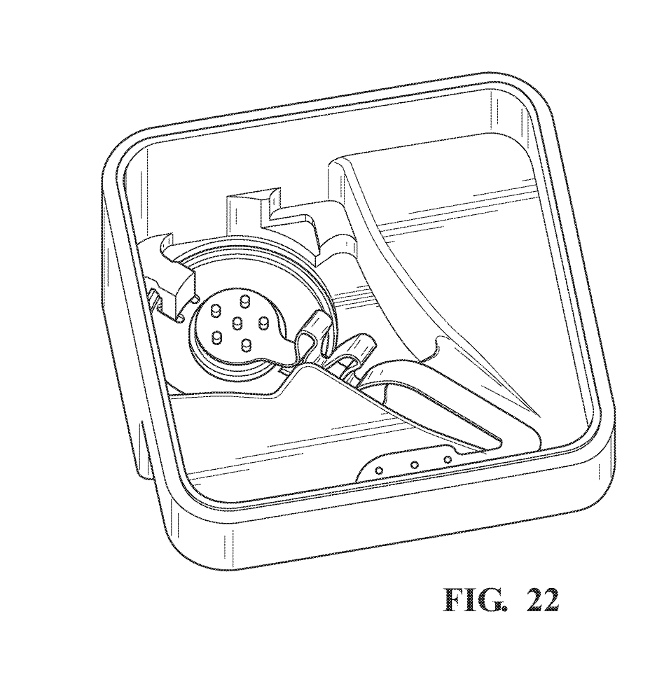

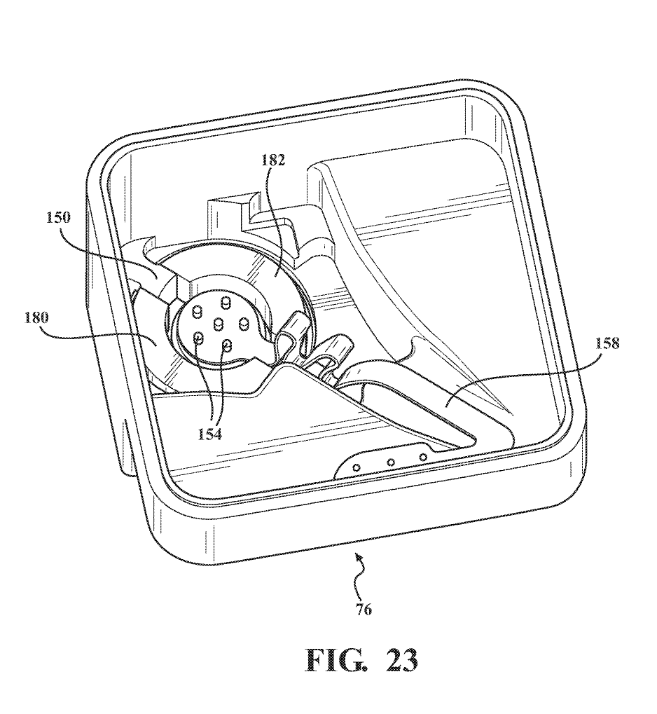

Referring to FIGS. 15-24, a connector 140 is fixed to the second housing 76 for connecting at least one of power and communication channels to the tracker 44. The second housing 76 has a main body 142 and a connector section 144 depending downwardly from the main body 142. The connector section 144 defines an opening 143 into which the connector 140 is located (see FIGS. 15 and 16). An inner counterbore 146 and an outer counterbore 148 are disposed radially outwardly from the opening 142 to support the connector 140. A post 150 and finger 152 project into the opening 143 to further support the connector 140.

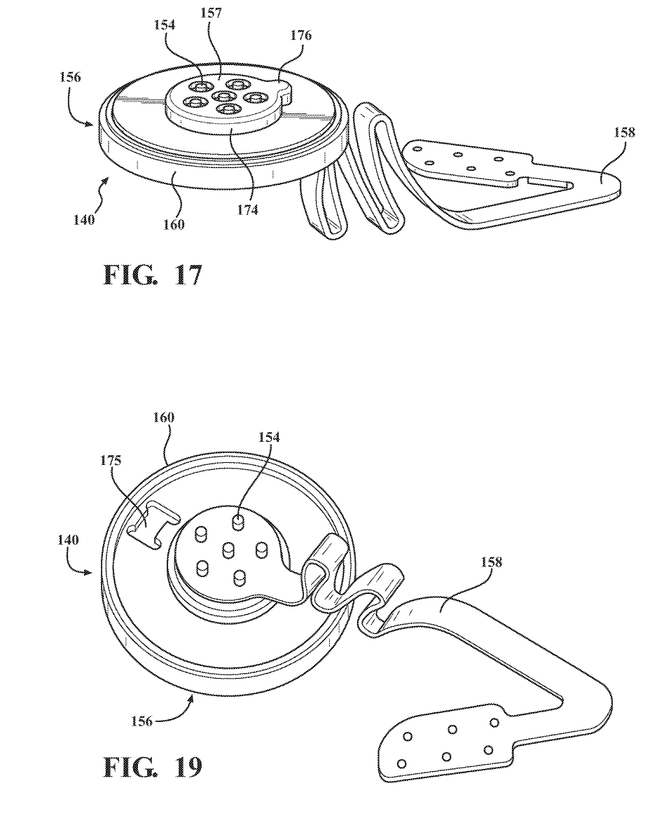

Referring to FIGS. 17-19, the connector 140 comprises a plurality of pins 154 formed at least partially of copper beryllium and, in some cases, completely of copper beryllium. The connector 140 also comprises a support structure 156 for the pins 154, a flex cable 158 to provide electrical communication between the printed circuit board PCB and the pins 154, and a bushing 160 formed of explosion bonded metals, such as stainless steel and titanium.

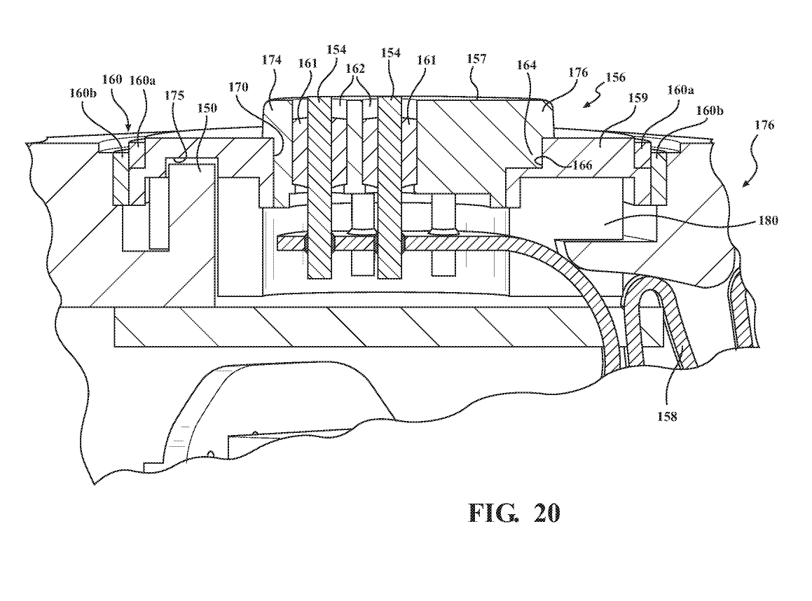

Referring to FIG. 20, the support structure 156 comprises a pin retainer 157 and an outer plate 159. The pin retainer 157 and the outer plate 159 may be welded together or formed in one piece. The pin retainer 157, in one embodiment, is generally cylindrical in shape and is formed of stainless steel, such as 304L stainless steel. The pin retainer 157 may assume different shapes in alternative embodiments. The outer plate 159, in one embodiment, is formed of stainless steel, such as 455 stainless steel. This material enhances the passing of magnetic flux through the outer plate 159, as described further below. In the embodiment shown, the outer plate 159, also referred to as a flux element, has a flat upper surface.

The pins 154 are sealed inside through passages 162 in the pin retainer 157 of the support structure 156 using polycrystalline ceramic 161, such as Kryoflex. The flex cable 158, which may be formed of any conductive material, is attached at one end to the pins 154 and the other end is coupled to the PCB. The pin retainer 157 of the support structure 156 has an orienting feature, such as a keyed portion 164, shaped to fit within a keyway 166 defined in the outer plate 159 so that the pin retainer 157 can be properly oriented relative to the outer plate 159.

The outer plate 159 also comprises a through opening 170 for receiving the pin retainer 157 so that a bottom of the pin retainer 157 and the outer plate 159 are coterminous. The pin retainer 157 further comprises an annular lip 174 that rests on the upper surface of the outer plate 159 when assembled. An orienting feature, such as a recess 175, is defined in a bottom surface of the outer plate 159 to receive the post 150 so that the outer plate 159 is properly oriented relative to second housing 76. The post 150 fits in the recess 175 so that the outer plate 159 is limited from rotation relative to the second housing 76.

The bushing 160 is explosion bonded or welded so that the bushing 160 is able to be welded to dissimilar metallic materials, such as stainless steel and titanium. For instance, by forming the bushing 160 of stainless steel (e.g., 304L stainless steel) and titanium materials that are explosion welded together, the titanium portion of the bushing 160 is able to be welded (e.g., hermetically laser welded) to the second housing 76, which is formed of titanium, and the stainless steel portion of the bushing 160 is able to be welded (e.g., hermetically laser welded) to the support structure 156, which is formed of stainless steel. As shown in FIG. 20, the bushing 160 has a first portion 160a formed of stainless steel and a second portion 160b formed of titanium.

A connector orienting feature 176, also referred to as a clocking feature, extends from the lip 174 to help orient a mating cable connector C from a tracker cable, as described further below. In the embodiment shown, the connector orienting feature 176 is a single projection shaped to receive a similarly shaped recess 177 in the cable connector C.

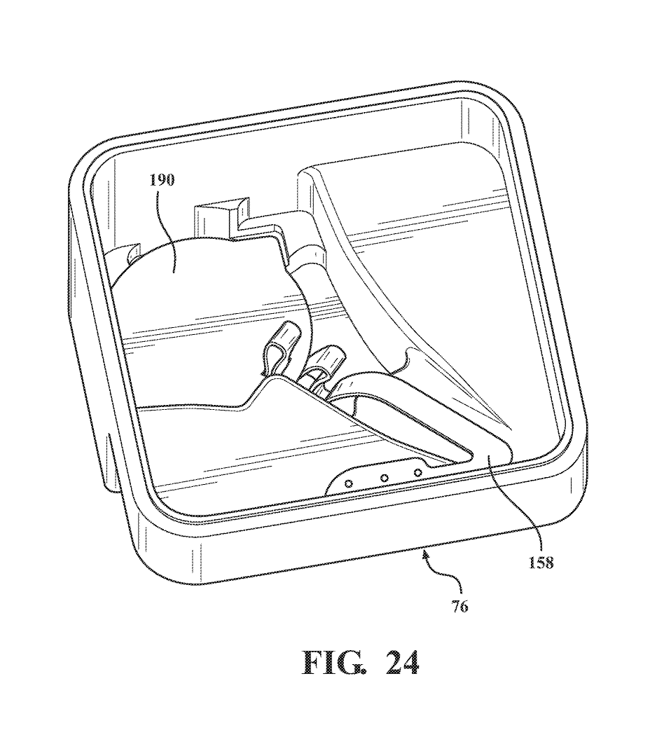

The connector 140 further comprises a pair of magnets 180, 182 to facilitate connection to the cable connector C. In one embodiment, the magnets 180, 182 are samarium-cobalt (SmCo) magnets. The magnets 180, 182 are arranged with opposing polarity facing toward the upper surface of the outer plate 159. A flux return plate 190 (see FIG. 24) is also provided for the magnets 180, 182 to direct the magnetic field toward the upper surface of the outer plate 159. The flux return plate 190 may be formed of mild steel and connected to the post 150 and the flex cable 158 as shown in FIG. 24.

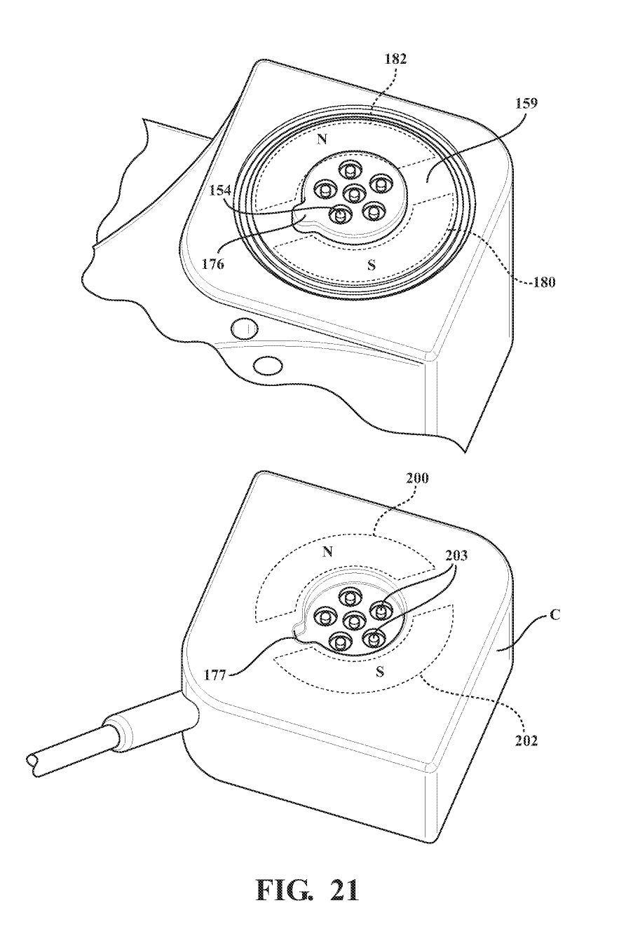

As shown in FIG. 21, the cable connector C has corresponding magnets 200, 202 also arranged with opposed polarity facing toward the upper surface of the outer plate 159 when properly connected. This magnet arrangement also facilitates proper orientation of the cable connector C to the connector 140 and prevents a user from improperly connecting the cable connector C to the connector 140. In other words, this magnet arrangement prevents the user from connecting the cable connector C to the connector 140 in a different orientation, in which case the pins 154 would not be aligned appropriately with the corresponding pins 203 on the cable connector C. Additionally, as previously mentioned, the outer plate 159 may be formed of 455 stainless steel to enhance the magnetic flux through the outer plate 159 and connection between the magnets 180, 182 and the magnets 200, 202.

Exemplary electrical schematics for the tracker 44 and for an error detection system for detecting errors in line-of-sight between the LEDs 50 and the optical sensors 40 are shown and described in U.S. patent application Ser. No. 14/156,856, filed Jan. 16, 2014, entitled, “Navigation Systems and Methods for Indicating and Reducing Line-of-Sight Errors” and published as U.S. Patent Application Publication No. 2014/0200621, hereby incorporated by reference herein.

Several embodiments have been discussed in the foregoing description. However, the embodiments discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

What is claimed is:

1. A tracking device for use with a navigation system to track an object, said tracking device comprising:
a tracking head;
a plurality of emitters supported by said tracking head, each of said emitters configured to emit light;
a plurality of lenses disposed over said plurality of emitters, each of said lenses having an outer wall with an arcuate inner surface and an arcuate outer surface spaced equidistantly from said arcuate inner surface to define a uniform outer wall thickness,
wherein each of said lenses are arranged relative to said emitters such that the light emitted from said emitters penetrate said outer wall normal to said arcuate inner surface to minimize refraction of the light.

2. The tracking device of claim 1, wherein each of said lenses has a dome shape.

3. The tracking device of claim 1, wherein each of said lenses are formed of sapphire.

4. The tracking device of claim 1, wherein each of said emitters comprise a die of an infrared light emitting diode.

5. The tracking device of claim 4, comprising a plurality of emitter support assemblies, each of said emitter support assemblies supporting one of said dies.

6. The tracking device of claim 5, wherein each of said emitter support assemblies comprises a reflector head for receiving said one of said dies.

7. The tracking device of claim 5, wherein each of said emitter support assemblies comprise a base and a pin that is sealed to said base with polycrystalline ceramic.

8. The tracking device of claim 5, wherein said lenses are soldered to said emitter support assemblies.

9. The tracking device of claim 5, wherein said tracking head comprises a first housing and a second housing connected to said first housing to define an internal chamber.

10. The tracking device of claim 9, wherein each of said emitter support assemblies are laser welded to said first housing.

11. The tracking device of claim 10, comprising a controller disposed in said internal chamber and hermetically sealed inside said internal chamber.

12. The tracking device of claim 10, wherein said first housing comprises a weld ring formed at least partially of titanium.

13. The tracking device of claim 12, wherein said first housing comprises a lid formed at least partially of titanium.

14. The tracking device of claim 13, wherein said first housing comprises a light ring disposed between said weld ring and said lid.

15. The tracking device of claim 14, wherein said lid and said weld ring are brazed to said light ring.

16. The tracking device of claim 14, comprising at least one light source arranged to emit light through said light ring.

17. The tracking device of claim 16, wherein said at least one light source comprises a light emitting diode configured to emit visible light.

18. The tracking device of claim 9, wherein said second housing is at least partially formed of titanium.

19. The tracking device of claim 18, comprising a connector for connecting at least one of power and communication channels to said tracking device.

20. The tracking device of claim 19, wherein said connector comprises a plurality of pins, a support structure for said pins, a flex cable, and a bushing formed of explosion bonded stainless steel and titanium welded to said second housing and said support structure.

21. The tracking device of claim 20, wherein said connector is welded to said second housing.

22. The tracking device of claim 20, wherein said connector comprises a pair of magnets configured to facilitate connection to a cable connector, said pair of magnets arranged with opposing polarity facing toward said support structure.

23. The tracking device of claim 22, comprising a flux return plate for use with said pair of magnets.

24. The tracking device of claim 1, comprising an antireflective coating applied to each of said lenses.

25. A navigation system for tracking an object, said navigation system comprising:
a tracking head;
a plurality of emitters supported by said tracking head, each of said emitters configured to emit light;
a plurality of lenses disposed over said plurality of emitters, each of said lenses having an outer wall with an arcuate inner surface and an arcuate outer surface spaced equidistantly from said arcuate inner surface to define a uniform outer wall thickness,
wherein each of said lenses are arranged relative to said emitters such that the light emitted from said emitters penetrate said outer wall normal to said arcuate inner surface to minimize refraction of the light; and
a localizer for receiving the light emitted from said emitters to determine a position and orientation of the object.

26. A method of manufacturing a tracking device for use with a navigation system to track an object, said method comprising the steps of:
assembling a plurality of emitters to a base at a predefined z-axis height relative to the base, each of the emitters configured to emit light;
locating a plurality of lenses over the plurality of emitters, each of the lenses having an outer wall with an arcuate inner surface and an arcuate outer surface spaced equidistantly from the arcuate inner surface to define a uniform outer wall thickness,
wherein each of the plurality of lenses are located relative to the emitters in an x-y plane such that the light emitted from the emitters penetrate the outer wall normal to the arcuate inner surface to minimize refraction of the light.
